(19) United States
(12) Patent Application Publication
(10) Pub. No.: US 2024/0238471 A1
(43) Pub. Date: Jul. 18, 2024

(54) FLUID STERILIZATION DEVICE FOR USE WITH UV LIGHT SOURCE

(71) Applicant: ORIGEN AIR SYSTEMS LTD., Victoria (CA)

(72) Inventors: Jonathan VAN ES, North Saanich (CA); Ryan LIDER, Victoria (CA); Sophia PENNER, Victoria (CA); Lucas ALGER, Victoria (CA); Andrew CRAWFORD, Victoria (CA)

(21) Appl. No.: 18/566,511

(22) PCT Filed: Jun. 1, 2022
(86) PCT No.: PCT/CA2022/050871

§ 371 Date: Dec. 1, 2023

(30) Foreign Application Priority Data
Jun. 2, 2021 (CA) 3120966

(51) Int. Cl. A61L 9/20

(52) U.S. Cl. CPC A61L 9/20; A61L 2209/111; A61L 2209/12; A61L 2209/14

(57) ABSTRACT

Described are various embodiments of a fluid sterilization device for use with a UV light source wherein the fluid flows through channels and the UV light source is disposed so as to sterilize at least a portion of the sterilization airflow volume in accordance with a designated spatial irradiance intensity profile and the airflow profile through the channels. The UV light source may be located near a region of higher fluid velocity in the housing as compared to other regions in the housing. The housing may include an inner divider separating the channels into an inlet channel and an outlet channel.

### RELATED APPLICATION

The instant application claims the benefit of priority to Canadian Patent Application serial number 3,120,966, entitled “FLUID STERILIZATION DEVICE FOR USE WITH UV LIGHT SOURCE”, and filed Jun. 2, 2021, the contents of which are herein fully incorporated by reference.

### FIELD OF THE DISCLOSURE

The present disclosure relates to fluid sterilization chambers, and, in particular, to a fluid sterilization device for use with UV light source.

### BACKGROUND

Environments are required where the air must be purified to provide a substantially sterile and contaminate-free supply of air. Such environments, for example, include environments when handling and packaging of food products and medicaments, when handling laboratory samples, surgical operations, and other environments where sterilized air is required or desired. The world COVID-19 pandemic has heightened this need and brought the need for sterilized air to more environments, such as the workplace, public facilities, and even one's own home where disease can be spread from one person to another. The pandemic has thus brought forth a need to produce sterilized air in more environments where, previously the need was much less.

Known in the art are various gaseous fluid purification systems, commonly used for the purpose of purifying air in various environments. Such systems intake air or gaseous fluids via an intake and then pass the gaseous fluid through one or more treatment zones so as to remove contaminants, particulates and/or sterilize the gaseous fluid, such as air.

For example, systems have been developed in which intake air is received into a purification system and then subjected to various treatment zones within the system to remove particulates via various filtering means and also to sterilize air by way of various means so as to destroy microorganisms, spores, and viruses or at least render pathogens incapable of reproduction and thus substantially sterilize air which exits from the system.

It is known that most biological contaminants and pathogens are carried on particulate matter present in air. Thus, should such pathogen-carrying particles not be filtered by particulate filters in a purification system, untreated pathogens in air exiting the purification system may still be able to cause infection to organisms in the surrounding environment. Therefore, ventilation and purification systems must also treat air with sterilization techniques before it exits the system for pathogens.

In order to address the need for ridding air from functional microorganisms and viruses, some devices provide germicidal qualities through the use of ultraviolet (UV) radiation produced by UV light sources. In order to minimize exposure of people, animals, and other organisms that may be adversely affected by UV radiation, air purification systems generally pass a fluid stream of air past the UV light source contained within an enclosure so as to inhibit UV emissions from escaping to a surrounding environment. Therefore, only the fluid stream of air passing the UV light source in such an enclosure is exposed to UV radiation. Accordingly, these systems provide various solutions to air purification as may be required or desired in certain environments.

Conventionally, UV or germicidal light sources are tubular and operate by providing an electrical discharge though argon gas which strikes mercury vapour to generate a photon with a wavelength of about 253.7 nm. Such mercury vapour UV lamps produce light in the spectrum range of about 180 nm to about 260 nm (i.e. UVC light). The effectiveness of UV light as a disinfecting agent also depends on the length of time that pathogens are exposed to the light and distance from the UV light source. Accordingly, the tubular mercury vapour UV light sources are generally placed in the enclosure lengthwise, along the travel path of the air fluid flow in the enclosure.

U.S. Pat. No. 6,939,397, entitled “System for Purifying and Removing Contaminants from Gaseous Fluids” and issued Sep. 6, 2005 to Nelsen et al., discloses a system that comprises an elongate enclosure having a gaseous fluid inlet and outlet where an elongate UV light source is disposed within the enclosure and oriented in the longitudinal direction along the length of the enclosure so as to increase the residency time that the gaseous fluid is in the enclosure and exposed to UV light. Therefore, due to the elongated nature of conventional mercury vapour UV-generating lights, air in the enclosure may pass along the length of the tubular bulbs and thus be exposed to the UV light for longer periods.

Canadian Patent number 2,415,997, entitled “Germicidal Lamp and Purification System Turbulent Flow” and issued Jul. 7, 2003 to Sauska et al., discloses a tubular UV light source that has a non-uniform contour, such as helix, formed into either the exterior surface of the tubular light source or on separate envelop enclosing the tubular UV lamp. The helical non-uniform contour is provided for creating turbulence of the fluid being passed over the UV light, generally in the longitudinal direction, in an enclosure which more evenly circulates pathogens to come into contact with the UV light and increases the residency time of exposure to the UV light. Therefore, a smaller enclosure may be used while maintaining the effectiveness of the UV light treatment of pathogens borne in the fluid.

International Patent Application number PCT/AU92/00221, entitled “Disinfectant System”, published Nov. 26, 1992 to Smith et al., discloses a system where there is a helical baffle located around a tubular UV light source. The tubular UV light source is oriented longitudinally in an enclosure such the fluid flow is lengthwise over the UV light source. The helical baffle is provided for inducing a laminar flow to the fluid over the UV light source and lengthen the path of the fluid flow over the UV light source, increasing the exposure time of the air to UV light.

Recent environment concerns have discouraged the use of mercury in various applications, such as tubular mercury vapour UV light sources. In fact, the U.S. Energy Policy Act of 2005 has banned the manufacture and sale of mercury vapour lighting for various applications, effective as of January 2008. Additionally, in 2013 the United Nations Environmental Programme introduced the Minamata Convention on Mercury which is an international treaty designed to control mercury emissions in several countries, with an aim to phase out several mercury products by 2020. Accordingly, an alternative UVC light source will ultimately be needed for fluid purification devices and systems. Light Emitting Diodes (LEDs) emitting UVC light (UV LEDs) have been developed that may be used in certain applications, and provide a potential avenue for replacing conventional tubular mercury vapour UV light sources.

However, in various applications, the eventual ban on the manufacture and sale of UV light-emitting tubular mercury vapour may be problematic. For example, air purification systems conventionally rely on the residency time of pathogen-containing air to provide an adequate sterility of treated air. To this end, UV LED sources may not be suitable for use with existing enclosures. For example, UV LED light sources are typically manufactured to provide directional light, rather than omnidirectional light like mercury vapour light sources. Therefore, in terms of air purification systems, UV LEDs may need to be placed within enclosures at locations where the UV light can be broadcast across the width or length of the enclosure to treat the air. In order to accommodate the use of UV LEDs, it may be desirable to design new enclosures for air purification systems using UV light treatment in order maintain purification efficiency comparable to conventional tubular mercury vapour lamp systems. Accordingly, the design of UV light enclosures may need to be reconsidered to adequately expose a given volume of air to UV light in order to effectively kill pathogens. Furthermore, it may be desirable to provide an enclosure for treating air with UV light from LEDs that can be utilised with other purification systems, such as particulate filters, HVAC systems, and/or other air purification components.

This background information is provided to reveal information believed by the applicant to be of possible relevance. No admission is necessarily intended, nor should be construed, that any of the preceding information constitutes prior art or forms part of the general common knowledge in the relevant art.

### SUMMARY

The following presents a simplified summary of the general inventive concept(s) described herein to provide a basic understanding of some aspects of the disclosure. This summary is not an extensive overview of the disclosure. It is not intended to restrict key or critical elements of embodiments of the disclosure or to delineate their scope beyond that which is explicitly or implicitly described by the following description and claims.

A need exists for a fluid sterilization device for use with UV light source that overcomes some of the drawbacks of known techniques, or at least, provides a useful alternative thereto. Some aspects of this disclosure provide examples of such systems and methods.

In accordance with one aspect, there is provided a device for sterilizing a flow of gaseous fluid comprising: an elongate housing having a quadrilateral perimeter shape with opposing first and second ends, the housing formed from a front panel, a rear panel, a first side panel, a second side panel, a curved bottom panel, and a top panel; an inner divider coupled to the first and second side panels and extending to near the curved bottom panel for partitioning the interior of the housing into an inlet channel and an outlet channel, wherein the inlet and outlet channels are in fluid communication, and the inner divider has a curved upper end coupled to an interior side of the front panel near the first end above a fluid inlet aperture for guiding fluid into the inlet channel; at least one fluid outlet aperture located near a downstream region of the outlet channel for allowing fluid to egress from the outlet channel; and at least one UV light source located near a region of higher fluid velocity in the housing as compared to other regions in the housing.

In one embodiment, the device further comprises at least one fluid current generating device located in the outlet channel near the downstream region of the outlet channel.

In one embodiment, the fluid current generating device is a fan.

In one embodiment, the at least one fluid outlet aperture is located in the top panel.

In one embodiment, the at least one UV light source produces UVC light.

In one embodiment, the at least one UV light source is located adjacent the inner divider in the inlet channel.

In one embodiment, the at least one UV light source is mounted to the inner divider.

In one embodiment, the at least one UV light source is located adjacent the rear panel in the outlet channel near the second end.

In one embodiment, the at least one UV light source is mounted to the rear panel.

In one embodiment, the at least one UV light source comprises an LED.

In one embodiment, the at least one UV light source comprises an LED array.

In one embodiment, the housing and the inner divider are reflective.

In one embodiment, the housing and the inner divider are aluminum.

In one embodiment, the inlet and outlet channels have a depth span of approximately 3 inches between the inner divider and the respective front and rear panels.

In one embodiment, the inlet and outlet channels have a width span of approximately 20 inches between the first side panel and the second side panel.

In one embodiment, the inlet and outlet channels have a depth span between the inner divider and the respective front and rear panels and a width span between the first side panel and the second side panel in a ratio of from about 0.1 to about 0.4.

In one embodiment, the inlet and outlet channels have a depth span between the inner divider and the respective front and rear panels and a width span between the first side panel and the second side panel in a ratio of about 0.15.

In one embodiment, one or more of the fluid current generating device or the at least one UV light source is configured to provide a variable output.

In one embodiment, the device further comprises at least one sensor configured to acquire data related to a device parameter.

In one embodiment, the at least one sensor comprises one or more of a temperature sensor, a humidity sensor, an anemometer, a particular matter sensor, an irradiance sensor, or a volatile organic compound sensor.

In one embodiment, the device further comprises a digital application associated with the device and operable to process that data related to a device parameter acquired by the at least one sensor.

In one embodiment, the digital application is operable to control one or more of the fluid current generating device or the at least one UV light source to provide an output in response to the acquired data related to a device parameter.

In one embodiment, at least a portion of one or more of an inner surface of the housing or the inner divider comprises a reflective medium.

In one embodiment, the reflective medium comprises a UV-reflective coating.

In one embodiment, the reflective medium is disposed on the portion to provide a designated energy distribution in a sterilization volume of the housing.

In accordance with another aspect, there is provided a device for sterilizing a flow of gaseous fluid comprising: an elongate housing having one or more sidewalls with opposing first and second ends, a curved bottom panel, and a top panel; an inner divider coupled to the one or more sidewalls, spanning from one side to the other, and extending to near the curved bottom panel for partitioning the interior of the housing into an inlet channel and an outlet channel, wherein the inlet and outlet channels are in fluid communication, and the inner divider has a curved upper end coupled to an interior side of the one or more sidewalls near the first end above a fluid inlet aperture for guiding fluid into the inlet channel; at least one fluid outlet aperture located near a downstream region of the outlet channel for allowing fluid to egress from the outlet channel; and at least one UV light source located near a region of higher fluid velocity in the housing as compared to other regions in the housing.

In one embodiment, the device further comprises at least one fluid current generating device located in the outlet channel near the downstream region of the outlet channel.

In one embodiment, the fluid current generating device is a fan.

In one embodiment, the at least one fluid outlet aperture is located in the top panel.

In one embodiment, the at least one UV light source produces UVC light.

In one embodiment, the at least one UV light source is located adjacent the inner divider in the inlet channel.

In one embodiment, the at least one UV light source is mounted to the inner divider.

In one embodiment, the at least one UV light source is located adjacent the one or more sidewalls in the outlet channel near the second end.

In one embodiment, the at least one UV light source is mounted to the one or more sidewalls.

In one embodiment, the at least one UV light source is mounted to the inner divider.

In one embodiment, the at least one UV light source comprises an LED.

In one embodiment, the UV light source comprises an LED array.

In one embodiment, the device comprises more than one UV light source.

In one embodiment, the housing and the inner divider are reflective.

In one embodiment, the housing and the inner divider are aluminum.

In one embodiment, the inlet and outlet channels have a depth span of approximately 3 inches between the inner divider and the one or more sidewalls near an apex of the sidewall.

In one embodiment, the inlet and outlet channels have a depth span between the inner divider and the one or more sidewalls near an apex of the one or more sidewalls and a chord between the edges of the one or more sidewalls defining a width span having a depth span to width span in a ratio of from about 0.1 to about 0.4.

In one embodiment, the inlet and outlet channels have a depth span between the inner divider and the one or more sidewalls near an apex of the one or more sidewalls and a chord between the edges of the one or more sidewalls defining a width span having a depth span to width span in a ratio of about 0.15.

In one embodiment, one or more of the fluid current generating device or the at least one UV light source is configured to provide a variable output.

In one embodiment, the device further comprises at least one sensor configured to acquire data related to a device parameter.

In one embodiment, the at least one sensor comprises one or more of a temperature sensor, a humidity sensor, an anemometer, a particular matter sensor, an irradiance sensor, or a volatile organic compound sensor.

In one embodiment, the device further comprises a digital application associated with the device and operable to process the data related to a device parameter acquired by the at least one sensor.

In one embodiment, the digital application is operable to control one or more of the fluid current generating device or the at least one UV light source to provide an output in response to the acquired data related to a device parameter.

In one embodiment, at least a portion of one or more of an inner surface of the housing or the inner divider comprises a reflective medium.

In one embodiment, the reflective medium comprises a UV-reflective coating.

In one embodiment, the reflective medium is disposed on the portion to provide a designated energy distribution in a sterilization volume of the housing.

In accordance with another aspect, there is provided a sterilization device for sterilizing a flow of air in an air purification system. The sterilization device comprises an airflow channel comprising a channel wall defining a sterilization airflow volume between an airflow inlet and an airflow outlet configured to allow respective air intake to and egress from the airflow channel, and the airflow channel is configured to impart a designated airflow velocity profile to the sterilization airflow volume. The sterilization device further comprises a UV light source disposed on an inner surface of the channel wall so to sterilize at least a portion of the sterilization airflow volume in accordance with a designated spatial irradiance intensity profile and the designated airflow profile. The airflow channel is configured for fluidic communication via the airflow inlet and the airflow outlet with an air intake of the air purification system configured to receive unsterilized air, and an air output of the air purification system configured to allow egress of sterilized air.

In one embodiment, the airflow channel of the sterilization device is further in fluid communication via one or more of the airflow inlet and the airflow outlet with an air purification module of the air purification system.

In one embodiment, the airflow inlet is configured to receive air intake from the purification module.

In one embodiment, the airflow inlet is configured to receive air intake from the purification module.

In one embodiment, the purification module comprises one or more of filter, a biofilter, or a genetically modified organism.

In one embodiment, the sterilization device further comprises an airflow current generating device.

In one embodiment, the sterilization device further comprises a UV-reflective medium disposed on at least a portion of the inner surface of the channel wall.

In one embodiment, the UV-reflective medium is disposed on the inner surface of the channel wall so to at least partially govern the designated spatial irradiance intensity profile.

In one embodiment, the UV light source is disposed on the channel wall in a region corresponding to a higher velocity of the designated airflow velocity profile as compared to another region of the sterilization airflow volume.

In one embodiment, the UV light source is disposed on the channel wall so to provide a uniformity of sterilization of air in the portion of said sterilization airflow volume for the designated spatial irradiance intensity profile and the designated airflow velocity profile.

Other aspects, features and/or advantages will become more apparent upon reading of the following non-restrictive description of specific embodiments thereof, given by way of example only with reference to the accompanying drawings.

### BRIEF DESCRIPTION OF THE FIGURES

Several embodiments of the present disclosure will be provided, by way of examples only, with reference to the appended drawings, wherein:

FIG. 1 is a top-front-right perspective view of an exemplary sterilization device, in accordance with one embodiment;

FIG. 2 is a top-back-left perspective view of the exemplary sterilization device of FIG. 1, in accordance with one embodiment;

FIG. 3 is a right-side perspective cut-away sectional view of the exemplary sterilization device of FIG. 1, in accordance with one embodiment;

FIG. 4 is a right-side cut-away sectional view of the exemplary sterilization device of FIG. 1, in accordance with one embodiment;

FIG. 5 is a right-side exploded perspective view of the exemplary sterilization device of FIG. 1, in accordance with one embodiment; and

FIG. 6 is a schematic representation of exemplary fluid flow velocities in a right-side sectional view of the exemplary sterilization device of FIG. 1, in accordance with one embodiment.

Elements in the several figures are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood elements that are useful or necessary in commercially feasible embodiments are often not depicted in order to facilitate a less obstructed view of these various embodiments of the present disclosure.

### DETAILED DESCRIPTION

Various implementations and aspects of the specification will be described with reference to details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Various apparatuses and processes will be described below to provide examples of implementations of the system disclosed herein. No implementation described below limits any claimed implementation and any claimed implementations may cover processes or apparatuses that differ from those described below. The claimed implementations are not limited to apparatuses or processes having all of the features of any one apparatus or process described below or to features common to multiple or all of the apparatuses or processes described below. It is possible that an apparatus or process described below is not an implementation of any claimed subject matter.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

In this specification, elements may be described as “configured to” perform one or more functions or “configured for” such functions. In general, an element that is configured to perform or configured for performing a function is enabled to perform the function, or is suitable for performing the function, or is adapted to perform the function, or is operable to perform the function, or is otherwise capable of performing the function.

It is understood that for the purpose of this specification, language of “at least one of X, Y, and Z” and “one or more of X, Y and Z” may be construed as X only, Y only, Z only, or any combination of two or more items X, Y, and Z (e.g., XYZ, XY, YZ, ZZ, and the like). Similar logic may be applied for two or more items in any occurrence of “at least one . . . ” and “one or more . . . ” language.

Unless defined otherwise, all technical and scientific terms used herein have the same meaning as commonly understood by one of ordinary skill in the art to which this invention belongs.

Throughout the specification and claims, the following terms take the meanings explicitly associated herein, unless the context clearly dictates otherwise. The phrase “in one of the embodiments” or “in at least one of the various embodiments” as used herein does not necessarily refer to the same embodiment, though it may. Furthermore, the phrase “in another embodiment” or “in some embodiments” as used herein does not necessarily refer to a different embodiment, although it may. Thus, as described below, various embodiments may be readily combined, without departing from the scope or spirit of the innovations disclosed herein.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

The term “comprising” as used herein will be understood to mean that the list following is non-exhaustive and may or may not include any other additional suitable items, for example one or more further feature(s), component(s) and/or element(s) as appropriate.

As described above, conventional sterilization systems typically rely on mercury lamps to provide UV light for fluid sterilization. Accordingly, various systems comprise form factors and/or structures that accommodate the tubular structure of commercial mercury lamps. While ultraviolet light emitting diodes (UV LEDs) are a potential means of providing sterilizing radiation, a need exists to develop sterilization system configurations that are able to address fluid flow rate requirements for different applications while also providing adequate sterility to fluids.

The systems described herein provide, in accordance with different embodiments, different examples of a sterilization device for use with a UV light source for purifying a fluid, such as air or water. Various embodiments of the exemplary sterilization systems described herein may manifest as standalone devices, for instance, for exposing a given volume of air to UV light in order to effectively kill pathogens (e.g. in a home or office space, in confined spaces, such as a car, plane, or bus, or the like). In accordance with other embodiments, such systems may be utilised in conjunction with, or as a component of, other purification systems. For example, and without limitation, embodiments of a sterilization system as herein described may comprise a sterilization component of a more comprehensive fluid purification apparatus, such as those known in the arts of, for instance, HVAC, HEPA or other particulate filtration systems, biological air purification systems (e.g. genetically modified plant-based biofilters), or the like.

In some embodiments, a sterilizing UV light source may be a UV LED, a plurality of UV LEDs, an array of UV LEDs, and/or a UV LED panel. For instance, an exemplary UV light source may comprise Klaran® Light Engine UVC LED array (e.g. systems comprising 3, 9, or 12 board-mounted UVC LEDs) for emitting UVC light predominantly between 260 nm and 275 nm, which may, in accordance with various embodiments, be conveniently incorporated within a sterilization system for on-demand sterilization. It will be appreciated, however, that various alternative UVC LED light sources and/or light source modules may be employed within a sterilization system, in accordance with different embodiments. For example, various UVC sources, in accordance with some embodiments, may primarily emit light with a wavelength at or near 253.7 nm, and may further produce light at additional wavelengths.

In accordance with various embodiments, various disinfection systems may be configured to provide both sufficient residence time and irradiance to particles within the system for sterilization (e.g. incapacitating and/or inactivating 98%, 99%, 99.9%, or the like, of viral, or other biological pathogens from air). For instance, and in accordance with various embodiments, sterilization efficacy may be a function of rate of irradiance of UV light on particulates, the distance from the UV source to particulates, and the residence time of particulates in irradiation zones. Accordingly, and in accordance with some embodiments, irradiance may be improved through minimising the average distance of particles from a UV or UVC light source, and/or encouraging particles to pass slowly through regions of high irradiance within the system, or otherwise increase the residency time of the particle exposure to the UV light. In some embodiments, this may comprise placement of one or more UVC LEDs at regions of low fluid velocity based on the particular configuration of the system. Conversely, various embodiments additionally or alternatively relate to the provision of a system configuration that produces desirable fluid flow conditions near a UV source.

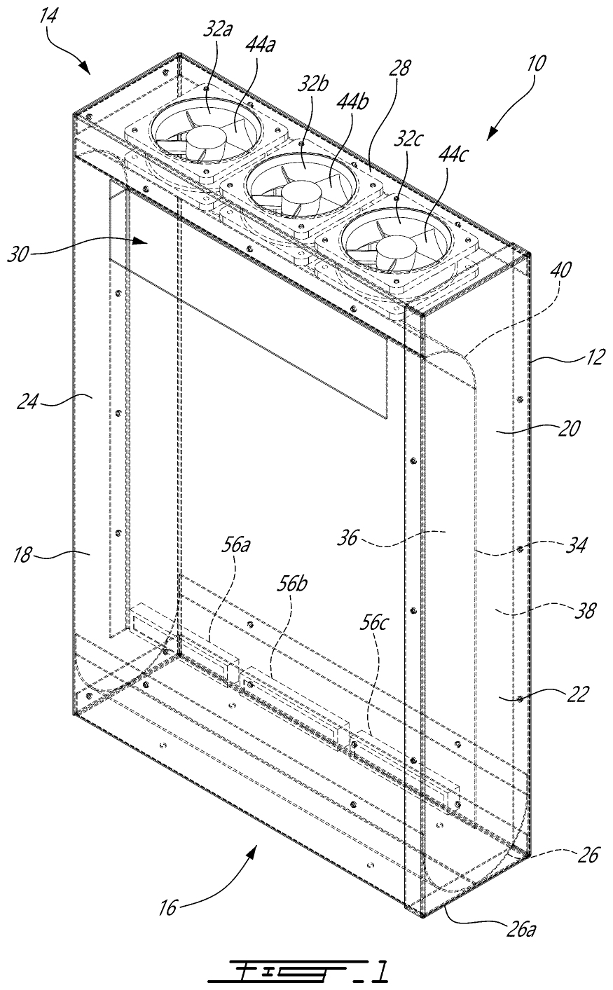

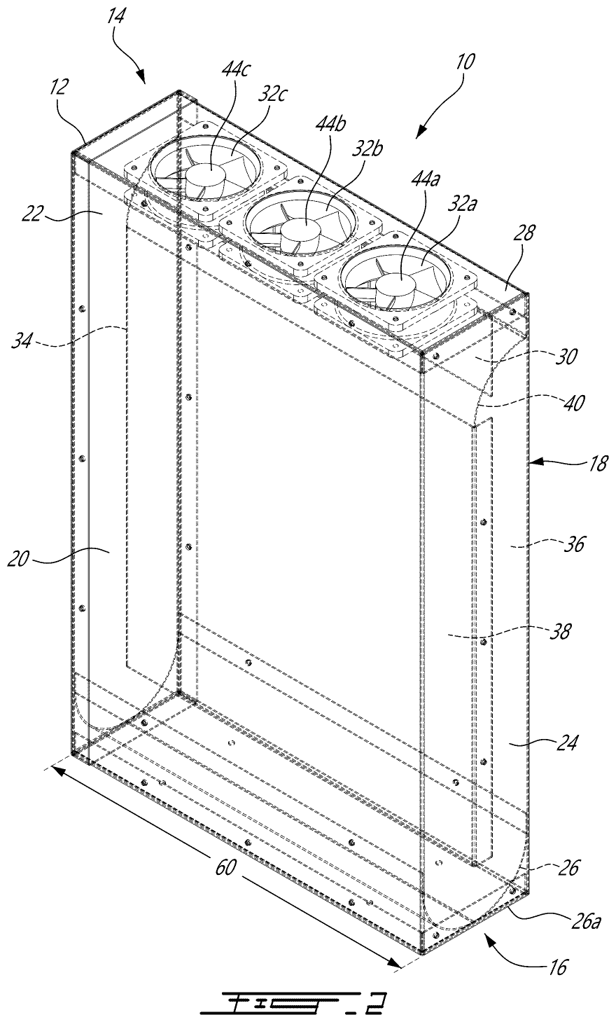

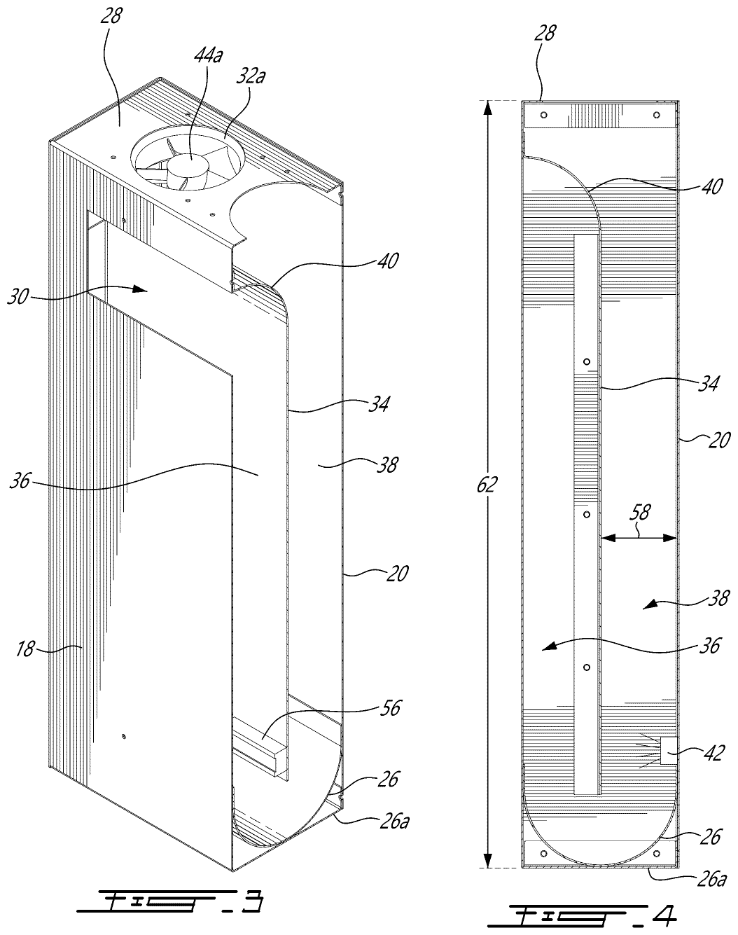

For example, FIGS. 1 to 5 schematically show different views of an exemplary configuration of a device 10 for sterilizing a flow of gaseous fluid, in accordance with various embodiments. In this non-limiting example the sterilization device 10 comprises an elongate housing 12 having a generally quadrilateral perimeter shape having a generally rectangular cross section (i.e. the housing is a cuboid). However, it will be appreciated that various embodiments relate to various housing 12 configurations, non-limiting examples of which may be generally described as cubical, cylindrical, or the like.

In accordance with various embodiments, the housing 12 may be generally described as having opposing first and second ends, respectively shown in FIG. 1 with the reference numerals 14 and 16. The housing 12 may further be formed from a front panel 18, a rear panel 20, a first side panel 22, and a second side panel 24. In accordance with various embodiments, the housing 12 may be enclosed with a curved panel 26, as will be further described below, and a top panel 28.

In the exemplary embodiment of FIG. 1, the curved panel 26 is disposed near the lower second end 16 of the device 10, and may accordingly be interchangeably referred to herein as a “curved bottom panel” 26. However, it will be appreciated that various embodiments relate to a device 10 having a similar housing 12 and relative disposition of elements to that exemplarily shown in FIG. 1, but is generally rotated. Accordingly, and in accordance with some embodiments, various configurations relate to a device that, when in use, may be inverted or otherwise rotated relative to the orientation schematically shown in FIG. 1. Similarly, the top panel 28 of FIG. 1 may, in different embodiments, serve as a base or side surface of the device 10, depending on the particular orientation of the device 10 when in use.

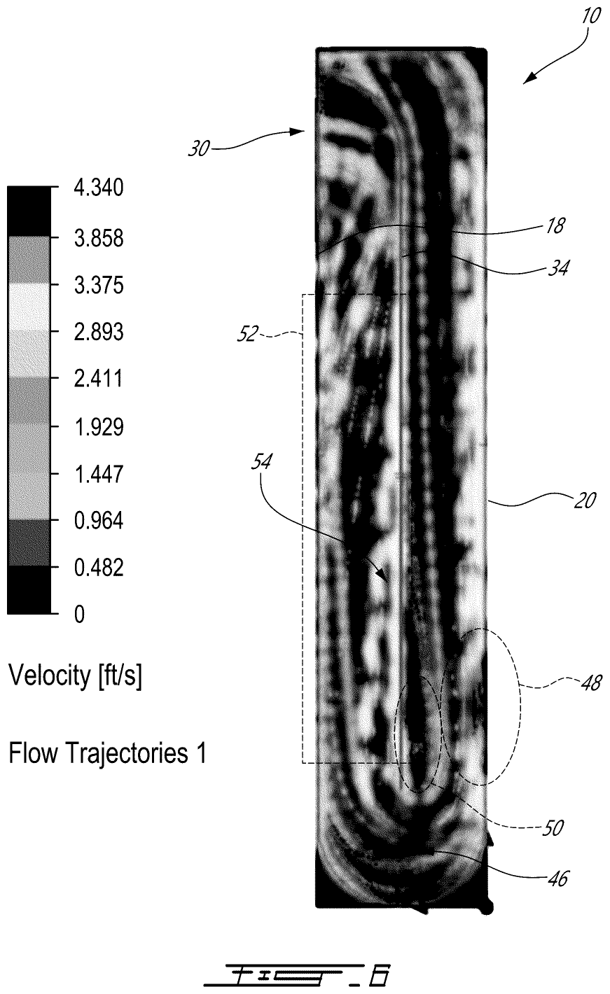

In accordance with some embodiments, the housing 12 may further comprise an enclosing panel 26a, generally disposed near the second end 16 of the device 10. This optional element may be included if, for instance, a flat surface is desirable at or near the second end 16, or if the housing 12 is, for instance, more easily manufactured be bending a planar material (e.g. aluminum sheet), or additional structural stability is preferred. For example, FIG. 5 schematically shows an exploded view of various components of the exemplary device 10, wherein the top panel 28, front panel 18, and bottom enclosing panel 26A are formed of a single piece of aluminum, or other suitable material, bent at 90° angles. However, and as will be further described below, the bottom enclosing panel 26A may be optional due to the presence of the curved bottom panel 26, which effectively encloses the second end 16 of the housing 12. The bottom panel may therefore not be included, in accordance with some embodiments, even if, for instance, the device 10 were to be inverted or in another orientation from that shown in the exemplary configuration of FIG. 1 when in use (i.e. the curved bottom panel 26 is disposed at the top of the device 10, such that the “top” panel 28 rests on a planar surface when in use), if the device 10 is supported by a different panel (e.g. coupled with an HVAC system via the front panel 18), or one or more of the front panel 18, rear panel 20, and/or first or second side panels 22 or 24, further comprise structures to support the device 10 (e.g. legs, wide bases, or the like).

In accordance with various embodiments, the housing 12 of a sterilization device 10 may comprise an inlet aperture 30. In the exemplary embodiment of FIG. 1, the inlet aperture comprises a rectangular aperture 30 in the front face 18 near the first end 14 of the of the housing 12, although it will be appreciated that various other aperture configurations are herein contemplated, in accordance with various embodiments. For example, the inlet aperture 30 may comprise a circular, oval, or other like geometry for allowing or guiding fluid (e.g. air) to enter the device 10. Similarly, an inlet aperture 30 may comprise an array of apertures 30 of similar or different geometries generally disposed at various positions in or near the first end 14 of the front face 18. For example, an inlet aperture 30 may comprise an array of 4×1 array of circular apertures disposed on the front face 18 of the housing 12 lengthwise parallel or perpendicular to the edge dividing the front face 18 and the top panel 28 near the first end 14 of the housing 12. In accordance with another embodiment, the inlet aperture 30 may comprise a nonlinear array (e.g. a 3×2 array) of apertures disposed in the front face 18 near the first portion 14 of the housing 12.

Similarly, the housing 12 of the exemplary embodiment of FIG. 1 further comprises fluid outlet apertures 32a, 32b, and 32c (also collectively referred to herein as a “fluid outlet aperture 32”). As described above with respect to the fluid inlet aperture 30, while the exemplary configuration of FIG. 1 schematically illustrates fluid outlet apertures comprising three circular apertures 32a, 32b, and 32c, various aperture configurations (e.g. one, two, four or more, or an array of apertures) will be appreciated as lying within the scope of the disclosure.

In accordance with various embodiments, the device 10 may further comprise an inner divider 34. While the exemplary embodiment of FIG. 1 shows a single inner divider 34, various embodiments may comprise a plurality of similar dividers, as further described below. Generally, an inner divider 34, in accordance with various embodiments, may be coupled to the first and second side panels 22 and 24 so to partition the housing 12 into a plurality of channels. For instance, the inner divider 34 of FIG. 1 extends from the interior side of the front panel 18 near the first end 14 of the housing 12 above the fluid inlet aperture 30.

In this embodiment, the inner divider 34 comprises a curved upper end 40 extending away from the front panel 18 and downwards towards the curved bottom panel 26. As the inner divider 34 is coupled to each side panel 22 and 24, but does not completely extend to the curved bottom panel 26, the housing 12 is effectively partitioned into two channels: an inlet channel 36 extending from the first end 14 to near the second end 16 of the device 10, and an outlet channel 38 extending from near the second end 16 to the first end 14 of the device. As the inner divider 34 does not extend completely to the curved bottom panel 26, the inlet channel 36 and the outlet channel 38 are in fluid communication with each other, effectively comprising a single serpentine channel that is longer than any segment thereof through which a fluid may flow.

In accordance with various embodiments, the device 10 may further comprise a fluid current generating device to generate a flow of fluid through the inlet and outlet channels 36 and 38 from the fluid inlet aperture 32 to the fluid outlet aperture 32. In the exemplary embodiment of FIG. 1, the fluid current generating device comprises three fans 44a, 44b, and 44c (also collectively referred to herein as a “fluid current generating device 44”) disposed in, respectively, the outlet apertures 32a, 32b, and 32c. Accordingly, in this embodiment, the fluid current generating device 44 is located at the downstream end of the outlet channel 38. It will be appreciated that while the following description relates to a sterilization device 10 comprising a fluid current generating device 44, various embodiments relate to a sterilization device 10 in which fluid current may be generated by an external source. For example, a sterilization device 10 may be operably coupled to a HVAC system providing a fluid flow through the sterilization device 10. Such a fluid current generating device 44 may thus draw fluid into the device 10, or pull the fluid through the device, or a combination of both. Similarly, various embodiments relate to a sterilization device 10 which are in fluid communication with an additional and/or alternative fluid purification system, such as a biofilter up- or down-stream from the sterilization device 10. In accordance with some such embodiments, a fluid current generating means, such as a fan, may be configured and/or in fluid communication with the additional purification system so to provide fluid flow through the sterilization device 10.

It will further be appreciated that a fluid current generating device 44 may comprise a means other than fans 44a, 44b, and 44c for generating a fluid current. For example, and without limitation, the fluid generating device 44 may comprise one or more turbines, pumps, or other means known in the art of, for instance, HVAC, liquid pumping systems, or the like so as to cause fluid flow through the device.

In the exemplary embodiments of FIG. 1, the fluid current generating device 44 is disposed within, or near, the fluid outlet aperture(s) 32. Such a configuration may be advantageous to, for instance, provide an accurate measure of exhaust flow. This may facilitate monitoring of the volumetric flow rate of a fluid exiting the chamber to, for instance, ensure that the fluid and/or particular matter therein receives adequate irradiation and/or exposure time for sterilization. This configuration may further, in accordance with various embodiments, provide a more stable or predictable fluid flow (e.g. flow rate, flow lines) than a configuration in which a fluid current generating device 44 is disposed at, for instance, only the fluid inlet aperture 30.

However, various alternate configurations are herein contemplated. While the configuration of FIG. 1 may be beneficial, for instance, for uniformly drawing fluid from through the inlet and outlet channels 36 and 38 from the fluid inlet aperture 32, various embodiments relate to the positioning of additional fluid current generating devices 44 to, for instance, increase a rate of flow through the inlet and outlet channels 36 and 38. For example, an additional fluid current generating device may further be disposed within the fluid inlet aperture 30. In accordance with other embodiments, an additional fluid current generating device(s) 44 may be disposed in the inlet channel 36, outlet channel 38, and/or a junction therebetween, or when used with other systems such as, for example, an HVAC system, a current generating device of the HVAC system.

When in use, the fluid current generating device 44 may effectively create a pressure differential that draws fluid from the inlet aperture 30 through the inlet channel 36 and outlet channel 38 before exiting the fluid outlet aperture 32 past the fluid current generating device 44. In this exemplary embodiment, the curved upper end 40 of the inner divider may guide fluid into the inlet channel 36 to, for instance, improve a flow of the fluid in the inlet channel 36. Similarly, the curved bottom panel may guide fluid flow from the inlet channel 36 to the outlet channel 38.

In accordance with different embodiments, the curved upper end 40 of the inner divider 34 and curved bottom panel 26 may comprise different curvatures or contours. For example, the curvature of these components in FIG. 1 has an arc approximating that of a portion of a circle. Such configurations may be beneficial to, for instance, reduce fluid friction losses throughout the device 10 and desirably shape air currents as compared to, for instance, a rectangular or square configuration. However, it will be appreciated that various other curvatures or geometries may be employed, in accordance with different embodiments. For example, it may be preferred for some applications and/or configurations to increase fluid friction in the device 10 via sharper curvature or rectangular bends in the upper end 40 of the inner divider 34 and/or bottom panel 26 to, for instance, provide a lesser flow rate, or to increase a turbulence of the system to improve sterilization properties (e.g. increase residence time, reduce the average particle distance from a UV light source, or the like).

For example, FIG. 6 is a schematic showing simulated air flow values throughout the inlet channel and outlet channels 36 and 38 of the exemplary device 10 of FIGS. 1 to 5. In this example, the system flow rate is 150 cubic feet per minute (CFM). For this configuration, fluid is generally flowing from the inlet aperture 30 and down through the inlet channel 36 before changing direction around a bend region 46 near the curved bottom panel 26 and flowing upwards through the outlet channel 38.

As shown in the spatial distribution of simulated velocity values for the configuration of the exemplary device 10 of FIG. 6, the highest fluid velocities values generally occur in the high velocity region 48 immediately upstream the bend region 46, near the rear panel 20 and above the curved bottom panel 26 in the outlet channel 38. Conversely, the lowest fluid flow velocities are generally found in the low velocity region 50 immediately upstream the bend region 46 and near the surface of the inner divider 34 opposite the outlet channel 38 from the high velocity region 48.

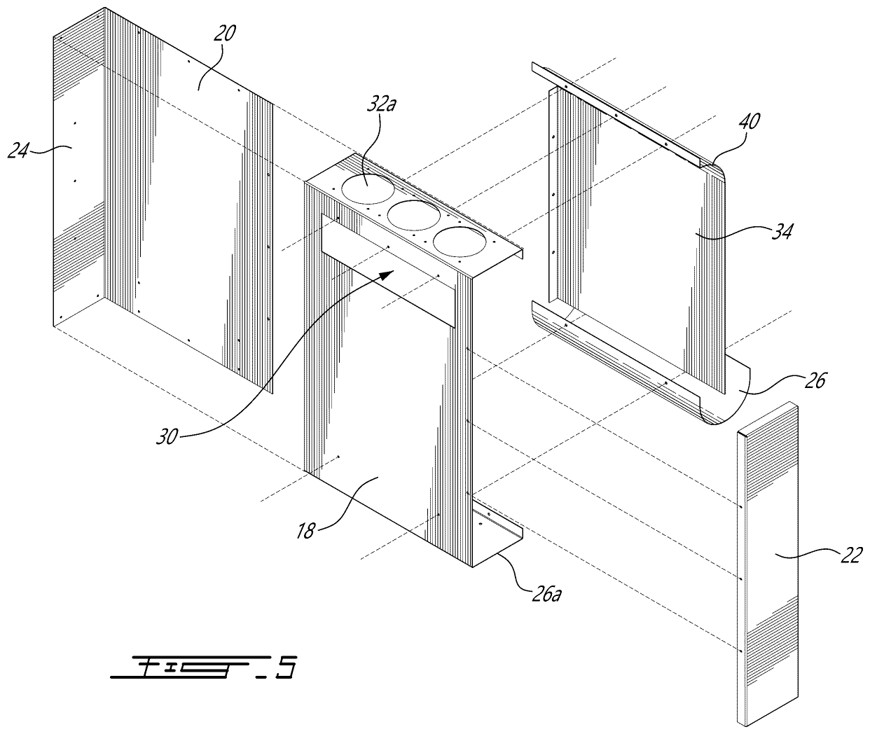

In accordance with various embodiments, the region generally defined by the high velocity region 48 and the low velocity region 50 may comprise a preferred location for a UV light source 42, such as an array of UV LEDs 42. For example, and in accordance with one embodiment, a UV light source 42 may be disposed on the rear panel 20 of the outlet channel 38, as shown in FIG. 4, in close proximity to the high velocity region 48 of FIG. 6 (i.e. on the surface of the rear panel 20 opposite the low velocity region 50, in the outlet channel 38). As radiation intensity decreases with distance from a light source, the location of UV LEDs 42 in FIG. 4 may thus enable strong irradiation of faster-moving fluid in the high velocity region 48 (i.e. fluid that will not reside in an irradiation zone for a long time), while more weakly irradiating slower moving fluid in the low velocity region 50, but for a longer duration. Accordingly, fluid flowing through the outlet channel past the UV light source may be more efficiently (e.g. uniformly) sanitized, as more weakly irradiated fluid has an increased residence time while fluid with a shorter residence time is more strongly irradiated.

While the UV light sources 42 are schematically illustrated in FIG. 4 as being disposed in a region of high flow velocity 48 opposite a region of slow flow velocity 50 in the outlet channel 38 for exemplary purposes, it may be preferred that a UV light source be disposed in a region of, for instance, the inlet channel 36 and/or outlet channel 38 in which fluid flow velocity is more moderate, further increasing irradiation time for particulates, or providing a more even distribution of irradiation across a channel cross section. For example, and with reference again to FIG. 6, the dashed box 52 schematically shows a flow region 52 comprising more moderate cross-sectional flow profile across the inlet channel 36 than the flow profile corresponding to the outlet channel across the high velocity region 48 and the low velocity region 50 of the outlet channel 38. Within the flow region 52, moderate fluid flow 54 is observed in the portion of the inlet channel 36 near the inner divider 34, while slower flow velocities are observed opposite the inner divider 34 in the inlet channel 36.

Accordingly, the surface of the inner divider 34 defining the inlet channel 36 and within the flow region 52 may comprise a preferred location on which a UV light source may be disposed. For example, and in accordance with various embodiments, FIG. 1 schematically shows a 3×1 array of UVC LEDs 56a, 56b, and 56c (collectively referred to herein as a “UV light source 56”) disposed on the inner divider 34 within the flow region 52. As described above, such a configuration may enable a more uniform irradiation profile across the inlet channel 36 in view of the relative shorter residence time of fluid near the inner divider 34 as compared to the fluid near the front panel 18 in the inlet channel 36.

In accordance with various embodiments, at least one UV light source 56 may therefore be disposed near a region of higher air fluid velocity in the housing as compared to other regions in the housing. For instance, for a given volume of a channel (e.g. flow region 52 of the inlet channel 36, a channel volume corresponding to an irradiation zone of a UV light source, or the like), a UV light source 56 may be located in a region characterised by a relatively higher flow velocity (e.g. high flow velocity region 48) as compared to another region of the housing (e.g. the slow flow velocity region 50) characterised by relatively slower fluid flow velocity.

Furthermore, in accordance with various embodiments, and as described above, various UV light source configurations may be employed within a sterilization device. For example, the flow profile across a cross section of the inlet channel 36 is well suited to the irradiation intensity profile emitted from a UV light source along a longitudinal length of the flow region 52 in FIG. 6. Accordingly, the array of UV light sources 56 in the inlet channel may alternatively comprise a panel of UVC LEDs 56 (e.g. tens to thousands of UVC LEDs 56), or may comprise LED module arrays (e.g. each LED of the UV source 56 may comprise a panel module comprising 12 UVC LEDs, such as a Klaran® Light Engine module). Furthermore, and in accordance with various embodiments, UV light sources 42 and/or 56 may be distributed throughout the device 10. For example, a plurality of independent UV light sources may be disposed throughout both the inlet and outlet channels 36 and 38, and/or different regions of the device 10 (e.g. near the bend region 46, near the inlet and/or outlet apertures 30 and 32, or the like) in order to increase the effective residency time of particulates and/or sterilization targets with respect to UV exposure. Furthermore, a UV light source for producing UVC light (e.g. one or more UVC LEDs) may further be disposed on one or more of the first and side panel 22 and 24, in accordance with some embodiments.

In accordance with yet other embodiments, UV light sources may be disposed in accordance with different sterilization configurations depending on an observed or predicted fluid flow for the configuration or application at hand. For instance, different device configurations (e.g. rectangular rather curved channel bends, sharper or less sharply curving bend regions, or the like) may exhibit different flow regimes, some regions of which may be well suited to UV light source placement as determined by, for instance, SolidWorks™ or like simulation systems, as exemplarily shown in FIG. 6. Accordingly, various embodiments relate to the provision of a UV light source based on, for instance, preferred flow regions as determined from such simulations. It will be appreciated that such simulations and/or configurations may be further employed to establish channel regions of laminar or turbulent flow which, in accordance with different embodiments, may be leveraged for different UV light source configurations depending on, for instance, the application at hand (e.g. desired throughput, surrounding environment, anticipated pathogen load, or the like).

In accordance with some embodiments, UV sterilization efficiency may be improved through the use of reflective surfaces. For example, while irradiation intensity typically decreases with distance from the light source, a reflective inner surface of the housing 12 may enable reflection of UV light back within a fluid channel. For example, the array of UVC LEDs 56 disposed on the inner divider 34 provide UV light most intensely in the inner channel 36 nearest the inner divider 34, while irradiation is relatively weaker within the inlet channel 36 nearest the front panel 18. However, and in accordance with various embodiments, a reflective inner surface of the front panel 18 may reflect radiation incident thereon back into the inlet channel 34, effectively increasing the amount of UV exposure in the inlet channel 36 nearest the front panel 18 as compared to what would otherwise be experienced by a fluid if the front panel 18 was not reflective. To this end, various inner surfaces of the housing 12 and/or inner divider 24 may comprise a reflective material, such as aluminum. Further, it will be appreciated that while various embodiments relate device 10 comprising a material that is inherently reflective, such as aluminum, various alternative embodiments relate to a housing 12 or inner divider 34 comprising a reflective coating (e.g. a reflective paint, a thin metal surface layer, or the like).

For example, it may be desirable for various aspects of a sterilization device to comprise and/or be made from base materials that are cost efficient and/or are amenable to facile or ready device fabrication, such as plastics, wood, or metals with suboptimal reflective properties. While potentially inexpensive and easy to work with, such materials may exhibit suboptimal reflective properties, or reduce the efficiency for designated wavelengths of light (e.g. UVC light). Not only may such aspects reduce efficiency of sterilization, as described above, they may also present various safety concerns, wherein, if unaddressed, potentially harmful radiation may otherwise escape the device.

However, and in accordance with some embodiments herein described, a sterilization device may comprise, for instance, one or more housing or device components (e.g. a front panel 18, a rear panel 20, a first side panel 22, a second side panel 24, an inner divider 34, or the like) having a base material that is not inherently highly reflective, or is not highly reflective to designated wavelengths of light, but comprises a medium, such as a coating or layer that exhibits one or more designated reflective properties. For instance, an inner divider (e.g. inner divider 34) or device surface (e.g. front panel 18) may comprise as a base material a plastics material or other material that inherently exhibits relatively low reflectivity to visible and UV light, but has disposed therein or thereon a medium that is highly reflective to UVC wavelengths. In some embodiments, while such a medium may be reflective to UV light, it may not necessarily be reflective to all wavelengths of light (e.g. visible light), although in other embodiments this may be the case. That is, in accordance with some embodiments, such a reflective medium may selectively reflect UV light, while, for instance, letting other wavelengths pass through. Such media may, of instance, comprise a coating or like material deposited on a substrate and/or device surface, and/or may comprise a material that is adhered onto a base material via, for instance, adhesive or other means.

For example, and in accordance with various embodiments, a sterilization device may comprise one or more surfaces or panels comprising a polytetrafluoroethylene (PTFE), Teflon, a variant thereof, or another medium that is UVC-reflective. In accordance with one non-limiting embodiment, this relates to at least one inner surface of a sterilization device comprising Porex Virtek® or like lining. However, it will be appreciated that other embodiments may comprise a similar additional and/or alternative medium that increase a reflectivity of a base material to one or more regions of the light spectrum (e.g. UV and/or UVC light), thereby improving sterilization efficiency and/or safety.

The employ of a reflective medium, coating, or lining may provide further advantages over conventional sterilization systems. For instance, and as noted above, the use of a UVC-reflective coating may enable the fabrication of a device using materials not traditionally suited to sterilization applications, such as plastics. The improved efficiency of such systems may further reduce down time and costs associated with device maintenance, whereby the improved efficiency of devices comprising UV-reflective surfaces provide improved resilience to dirt or other contaminants (e.g. a sterilization device may maintain adequate sterilization characteristics upon prolonged exposure to contaminated fluid, requiring less frequent cleansing of the device). In accordance with some embodiments, such reflective media may further be flexible and/or lightweight, facilitating the fabrication, installation, and operation of sterilization devices. Using, for instance, a PTFE-based UV-reflective coating increasing the energy efficiency of a UV air purifier may further increase the efficiency of germicidal radiation for sterilizing air containing microorganisms having a relatively high resistance to UV radiation. Further, by improving the efficiency of sterilization through the employ of reflective media, fewer UV light sources, or operation thereof at reduced intensity settings, may reduce the cost of device fabrication and operation, for instance the through reduction of the number of UVC LEDs that must be employed to achieve a designated sterilization dose, or the reduction power consumption of the system. For instance, fewer LEDs may be more distantly spaced apart in a sterilization device having more efficient UV propagation and reflection, allowing for an airflow to be irradiated over longer distances for a given number of LEDs.

Moreover, in addition to generally improving a sterilization efficiency as compared to, for instance, an aluminum surface, a UVC-reflective material may be selectively disposed at one or more regions of a sterilization device to provide designated sterilization characteristics within one or more designated flow regions. For example, while one embodiment relates to generally lining the inner surface(s) of a sterilization housing, other embodiments relate to the selective placement of a UVC-reflective medium to one or more designated regions of an inner surface of a sterilization device based on, for instance, a particular sterilization application and/or system configuration.

That is, based on, for instance, the particular sterilization device configuration (e.g. the configuration of a flow channel, such as the inlet and outlet channels 36 and 38 of the device 10), sterilization may be most efficient in a particular channel region(s) based on fluid flow within a given channel geometry and a given sterilization source placement (e.g. the disposition of a UVC light source within a channel). Accordingly, a UVC-reflective coating may be selectively disposed within the channel and/or on a surface thereof to influence an irradiation profile to achieve, for instance, a minimum designated irradiance level for a given channel cross-section.

Similarly, a particular channel configuration may be preferred based on, for instance, a desired flow rate through the device, or a fluidically coupled purification system (e.g. a biofilter). However, in such cases, and in accordance with some embodiments, it may not be necessary, for a particular application, to irradiate the entire channel volume. Accordingly, selectively enhancing UVC reflectivity in a designated channel region may provide sufficient irradiation levels for sanitation while reducing the costs of device fabrication (e.g. via a reduced area of reflective coating and/or UVC-opaque materials employed to meet safety standards) and energy use required for sterilization within overall channel constraints or preferences.

Similarly, a reflective coating may be employed to generally shape an irradiation profile produced by one or more light sources. For example, while light intensity from a UVC LED may dissipate with distance from the source, a UVC-reflective coating may be selectively disposed relative to a light source to effectively shape a beam of light emanating therefrom. For example, a UVC LED or array thereof may be disposed on an inner surface of a sterilization device such that it is partially or completely surrounded by or adjacent to a device portion having a UVC-reflective coating. Light from the UVC LED may then be more efficiently projected within a channel volume defined by the configuration of the light source and adjacent reflective material. Accordingly, an irradiation profile geometry may be controlled or dictated, for instance to provide a high-intensity ‘slice’ or ‘blade’ of irradiation volume across a channel and originating from one or more light sources. Such a designated irradiation intensity profile (e.g. designated based on a system configuration, such as a channel width, a fluid flow rate, a desired residence time, or the like) may similarly be established and/or controlled via alternative light shaping means, non-limiting examples of which may include mirrors, lenses, or other beam shaping elements known in the art.

As an irradiation intensity profile may be defined to achieve a desired sterilization characteristic through the use of a UV light source and one or more light directing elements (e.g. reflective coatings, mirrors, or the like), some embodiments herein described may be similarly related to the provision of a light diffusivity profile. For example, light shaping elements may be disposed and/or control in view of a device geometry to diffuse light, which may be of benefit in various applications. For example, one embodiment comprises an inner surface of the device having disposed thereon a UV-reflective coating characterised by a high reflectivity of UV light, wherein the coating is configured to provide a diffuse and/or uniform energy distribution within a sterilization volume.

It will be appreciated that such aspects, in accordance with various embodiments, may facilitate or enable various unconventional device configurations, and may similarly enable the use of sterilization devices such as those herein described for various diverse applications. For instance, various embodiments relate to the sterilization of air in a business space, such as a home office or office building. While traditional sterilization systems may be limited to, for instance, stand-alone sterilization device configurations excessively consuming energy to provide adequate sterilization with low-efficiency light sources and/or flow rate control, various embodiments herein described allow for highly efficient fluid sterilization with a minimal spatial and energetic footprint.

For example, and without limitation, various embodiments may be applied within light fixtures, vents, or the like, of an office space or home, wherein flowing fluid (e.g. air flow produced by a fan or like fluid current generating means, air circulating naturally through convective forces, or the like) may be generally directed through an irradiated sterilization volume. Through increased efficiency of sterilization through, for instance, disposition of a light source in a region of fluid flow that is greater in velocity than that of another region of the volume to be sterilized, the use of UVC LEDs, the use of UVC-reflective surfaces, and/or the shaping of an irradiation intensity profile through the use of beam-shaping elements, such as device regions comprising a UVC-reflective coating, lenses, mirrors, or other beam shapers, fluid sterilization may be provided where conventional approaches may fail to achieve an adequate degree of efficiency, in accordance with various embodiments.

In accordance with various embodiments, fluid flow within a sterilization device may be influenced by the device configuration. For instance, flow velocity profiles such as that of FIG. 6 are in part a function of device and component dimensions. In this exemplary embodiment, the inner divider 34 partitions the housing 12 into inlet and outlet channels 36 and 38 each having a respective depth span 58 of approximately 3 inches (i.e. the inner divider 34 is approximately 3 inches from both the front and rear panels 18 and 20). Indeed, various embodiments relate to a device comprising channels having a similar depth span 34 so to provide a high degree of irradiation from a UV light source across the width of a channel. It should be noted that one of skill in the art may determine that other depth spans in certain applications may be appropriate.

The exemplary embodiment of a sterilization device 10 in FIGS. 1 to 6 further comprises a width span 60 of approximately 20 inches between the first side panel 22 and the second side panel 24 (i.e. the inlet channels 36 and 38 comprise a width 60 of approximately 20 inches). Accordingly, the inlet and outlet channels 36 and 38 of the device 10 may be characterised as having a depth span 58 between the inner divider and the respective front and rear panels 18 and 20 and width span 60 between the first side panel 22 and the second side panel 24 in a ratio of about 0.15. In accordance with various embodiments, such a configuration may be preferred to provide a high degree of irradiation from a UV light source (e.g. UV LEDs 42 and/or 56) while maintaining sufficient volumetric flow for many applications (e.g. for use in a biological air purifier). However, it will be appreciated that the configuration of device 10 is described for exemplary purposes, only, and that various other embodiments may relate to sterilization devices characterised by different channel depths 58, widths 60, or ratios thereof. For example, various embodiments relate to sterilization systems comprising configurations wherein the inlet and outlet channels 36 and 38 have a depth span 58 between the inner divider 34 and the respective front and rear panels 18 and 20 and a width span 60 between the first side panel 22 and the second side panel 24 in a ratio of from about 0.1 to about 0.4.

Furthermore, it will be appreciated that while the inner divider 34 partitions the housing 12 into inlet and outlet channels 36 and 38 having equal depths 58, various embodiments further related to a sterilization device in which the inlet and outlet channels 36 and 38 comprise different widths. For example, the device 10 of FIGS. 1 to 6 comprises an inlet and outlet channels 36 and 38 having respective depths 58 of 3 inches. However, various other embodiments may relate to a device 10 comprising an inlet channel 36 having a depth of 2 inches and an outlet channel 38 having a depth of 4 inches. It should be noted that one of skill in the art may determine that other, or varying, depth spans in certain applications may be appropriate. For example, a sterilization device employed in the context of a HVAC system for an office building may have different dimensions (e.g. depth span 58 and width span 60) than a sterilization system employed in a biological purification system.

Such configurations may be advantageous in, for instance, providing desirable flow rates and/or profiles within respective channels of the device 10. For example, the flow region 52 of FIG. 6 corresponds to a total residence time of particulates flowing therethrough of approximately 1.75 seconds when three fans 44a, 44b, and 44c are operated at respective speeds corresponding to a total of 150 CFM (i.e. each fan contributing a volumetric flow rate of approximately 50 CFM), in accordance with one embodiment. However, it may be preferred for a particular application, form factor, or configuration of a sterilization device that fluid flow through the flow region 52 of the device at a slower rate (e.g. to maximize residence time in a channel region that has a preferred flow profile cross section). Accordingly, a device 10 may comprise a deeper inlet channel 36 (e.g. a depth 58 of 4 inches) so to provide a relatively slower flow rate through the inlet channel 36 for a given fan speed, thereby increasing residence time in the flow region 52, in accordance with various embodiments.

Conversely, a flow rate through the device 10 may be controlled through the moderation of fan speeds. For instance, a fan speed may be decreased in order to increase residence time of pathogens within the system, thereby increasing a degree of sterilization. Alternatively, if less residence time is required for a particular application, or to increase a flow rate through the system to increase throughput, higher fan speeds may be used. For example, while the residence time of fluid in the flow region 52 of FIG. 6 is 1.75 seconds for a fan speed corresponding to 150 CFM, the residence time for fluid in the same region 52 is increased to 5.25 seconds for a fan speed corresponding to 50 CFM, in accordance with another embodiment.

Residence time of fluid within a sterilization system, in accordance with various embodiments, may further be a function of channel length. For example, the inlet and outlet channels 36 and 38 of FIGS. 1 to 5 have a length span 62 corresponding to a front panel length of approximately 30 inches. Various embodiments, however, relate to the use of longer or shorter inlet and outlet channels 36 and 38 which may, respectively, increase or decrease a residence time of fluid within the system for a given fluid current (e.g. a current generated for a particular fan speed). Further, embodiments relating to a sterilization system comprising a longer length span 62, or in some embodiments a wider width span 60 in some regions along the length span of the device, may provide, for instance, additional space real estate within the system on which UV light sources may be disposed for increased sterilization and/or increase the residency time of the fluid being exposed to the UV light. Accordingly, one of skill in the art may determine that a certain channel length is appropriate for a given application.

While the embodiments of FIGS. 1 to 6 schematically show a sterilization system comprising an inlet channel 36 and outlet channel 38 in fluid communication via a bend region 46, various embodiments further relate to a housing 12 comprising additional interior channels. For example, various embodiments relate to a sterilization system further comprising an additional inner divider extending from near the second end (e.g. from the bottom enclosing panel 26a) to near the top panel 28, thereby further partitioning the housing 12. Such a configuration may therefore effectively increase the overall inner channel length via formation of a serpentine channel through which fluid flows (i.e. from the inlet aperture 30, through the inlet channel 36, the inner channel, and the outlet channel 38, and exiting through the outlet aperture 32). It will be appreciated that such a configuration may further comprise a top curved panel (e.g. similar to the curved bottom panel 26 but disposed near the first end 14 of the system) to guide fluid through the serpentine channel. It will further be appreciated that in such a configuration, the fluid inlet aperture 30 and fluid outlet aperture 32 may be disposed at different ends of the device 10. For example, in a device comprising two inner dividers, an inner serpentine channel may comprise three segments. Accordingly, if the inlet aperture 30 is disposed near the first end 14 of the device 10, then the outlet aperture 32 may be disposed near the second end 16.

It will further be appreciated that any number of inner dividers and corresponding curved panels may be included within a device to increase a serpentine channel length. For a device comprising an odd number of inner channel segments, it may be preferable for the inlet and outlet apertures 30 and 32 to be disposed near different ends of the device, so to more fully utilise the overall channel length for sterilization (i.e. provide high residence times). Conversely, for a device comprising an even number of inner channel segments, it may be preferable for the inlet and outlet apertures 30 and 32 to be disposed near the same end of the device 10, as shown in FIGS. 1 to 6. It will be appreciated that for such configurations, fluid current generating devices may be increased in number or output (e.g. higher fan speeds) to provide a desired fluid output. The increase in inner surface area of the device (e.g. the additional inner divider surfaces), however, may provide additional regions for UV light source placement, further increasing the sterilization ability of the device.

It will be appreciated that for a given channel configuration (e.g. a particular depth span 58, width span 60, and length span 62, number of inner channels) a fluid current generating device 44 may be operated so to increase or decrease a fluid flow rate to decrease or increase, respectively, a residence time of fluid within one or more regions of a sterilization device. For example, a fan speed may be increased to obtain a larger clean air delivery rate (CADR). Similarly, UV light sources may be operated for a given fluid flow rate to provide, for instance, a target sterilization percentage (e.g. 99.9%, or 99.9999% of pathogens destroyed). Accordingly, various embodiments of a sterilization system comprising a UV light source and fluid current generating means may comprise various configurations while maintaining, for instance, a log-reduction in pathogens while simultaneously maintaining a target clean air delivery rate.

In accordance with various embodiments, various components of a sterilization device 10 may be automated, or remotely controlled. For instance, one or more sensors may be disposed on or otherwise incorporated within a sterilization device 10, such as a flow meter sensor or anemometer (e.g. disposed near the outlet aperture 32 to measure, for instance, a fluid flow output, near the input aperture, and/or in the inlet or outlet channels 36 and 38), a sensor figured to sense a volatile organic compound (VOC), a particulate counter or particulate matter sensor (e.g. a PM 10 sensor, a PM 2.5 sensor, or the like), an irradiance sensor, a temperature sensor, a humidity sensor, or the like. Such sensors may, in accordance with various embodiments, be in network communication with one or more processors and/or digital applications operable to, for instance, monitor sensor values in real time.

Similarly, various sterilization device 10 components may further be in network communication with such a digital application from which remote control may be enabled. For instance, a fluid current generating device 44 may be remotely controlled (e.g. over a network) in response to, for instance, fluid flow meter readings to increase or decrease a fan speed to increase or decrease a throughput of fluid in the device, in accordance with some embodiments. Further, various embodiments relate to a device in which such fluid current generating devices 44 may be operated in various modes as instructed via, for instance, a digital application or manual control associated therewith. For instance, the device 10 may comprise a first operational mode in which the current generating device 44 generates a fluid flow rate of 150 CFM, resulting in a particular residence time of 1.75 seconds in the flow region 52. Upon instruction from, for instance, a digital application associated with the system, or in response to an increase particulate count as determined by, for instance, a sensor monitoring the same and associated with system, the fluid generating device 44 may be remotely and/or automatically reduced in output to in a second operational mode (e.g. fan speeds reduced to 50 CFM) to increase residency time in the flow region 52. Such modes may be enabled depending on, for instance, anticipated pathogen load, or for a particular application.

For example, and without limitation, a fluid current generating device 44 may comprise an operational mode corresponding to total fluid residence time within the inlet and outlet channels 36 and 38 of 1 minute (e.g. for particularly high pathogen loads). Another operational mode may comprise a total fluid residence time of approximately 10 seconds. It will be appreciated however, that such flow rates may be a function of, for instance, a particular UV light source configuration, intensity, and/or profile through the device 10. For example, while one embodiment relates to a UV light source configuration for which a preferred residency time of fluid within the system is approximately 20 seconds to approximately 30 seconds. However, an alternative embodiment relates to a sterilization system comprising a higher UVC LED count that may efficiently sterilize a fluid with a reduced residence time.

In accordance with yet other embodiments, a UV light source within the system (e.g. UV sources 42 and/or 56) may be in network communication with a digital application or control system (e.g. wireless communication with an external server or computing device, or a like system on-board the sterilization device itself). For example, a UV light source, a sensor monitoring an output or performance thereof, may report a status associated with one or more of the UV sources of the device. For example, and without limitation, a UV source or sensor may communicate to a digital platform associated therewith if a change in output is observed, such as if a UV LED is producing sub-optimal or reduced output, if it has died, needs replacement, or the like. Accordingly, various embodiments relate to the sensing a property related to individual UV light sources within the device. In accordance with other embodiments, such sensor data relate to a plurality or collection of UV sources, such as the total irradiance received at a designated position within a device channel from an array of UV LEDs.

In accordance with other embodiments, a UV light source within the device 10, or a plurality of UV light sources, may be controlled via a digital application. Some embodiments, for instance, relate to the independent and/or automatic control of different UV light sources within a sterilization device. For example, a digital application associated with the device (e.g. within a computing system mounted on the device 10, or in network communication therewith), may be operable to activate, deactivate, and/or tune one or more UV light sources of a sterilization device. Such control may, in some embodiments, be automatically or remotely controlled in response to data received from sensors associated with the device, such as those described above with respect to UV light output and/or intensity, or a measure of particular matter prior to entry to, during transit through, or upon exit of the sterilization device. In accordance with other embodiments, such sensor data may relate to, for instance, the housing 12 of the device 10 itself. For example, as UV light escaping the device may pose a risk to an organism in a surrounding environment, a sensor operable to detect an opening in the housing 12 (e.g. a circuit configured to provide a signal in response to the opening of a door or panel of the housing 12) may trigger the automatic shut off of a UV light source within the device. As described above with respect to fluid current generating devices 44 within the system, a UV light source(s) may similarly comprise various operational modes that may be provided based on, for instance, an anticipated particulate load, a desired irradiation level and one or more positions within the device, or the like.

Accordingly, and in accordance with various embodiments, one or more of a UV light source or a fluid current generating device may be operable to provide variable output. For example, a fan may be operable to provide fluid currents in accordance with different fan speeds. Similarly, a UV light source may be operable to output different irradiance strengths or profiles. For example, a UV light source may comprise an array of independently addressable UV sources (e.g. UVC LEDs). In accordance with some embodiments, individual sources may be independently activated/deactivated to increase/decrease irradiance in the system, and/or to spatially control where fluid is irradiated in the system. In accordance with some embodiments, such variable output may be provided in response to, for instance, sensor data acquired from one or more sensors associated with the sterilization device.

In accordance with various embodiments, one or more elements of a sterilization device may be removable. For example, and without limitation, the inner divider 34 may be reversibly coupled (e.g. via screws, or other reversible fastening device known in the art) to the front, first side, and second side panels 18, 22, and 24 so to enable removal to, for instance, repair a damaged UV source 42 or 56. Similarly, and in accordance with some embodiments, one or more housing 12 components (e.g. the front panel 18) may be removable to allow access to various regions of the housing 12. In accordance with yet other embodiments, the housing 12 may comprise a door or other structure (e.g. a door in the rear panel 20) so to facilitate access to, for instance, UV light sources within the device 10.

It will be appreciated that the configuration of the sterilization device 10 of FIGS. 1 to 6 is provided for illustrative purposes, only, and that various other configurations are hereby considered, in accordance with other embodiments. For instance, a fluid flow channel may be defined by a housing and/or dividing surface(s) to provide generally different flow patterns and/or flow regimes from those described above with reference to FIGS. 1 to 6, including, and without limitation, fluid flow through approximately circular, oval, or spiral channels, through channels having one or more vertices (e.g. generally square or corner-comprising geometries), or a combination thereof.

For example, and without limitation, various embodiments herein described relate to the sterilization of a fluid volume in concert with purification from one or more biofilters (e.g. plant species or variants thereof removing one or more contaminants from air). In such embodiments, a fluid sterilization device, as herein described, may be disposed up- or down-stream of such complementary systems, and may accordingly be configured to communicate fluid therewith in accordance with a designated or preferred fluid flow regime. For example, one embodiment relates to a sterilization device disposed atop an air purification system, wherein fluid from the complementary system is received as intake from an intake disposed on a lower or bottom portion of the device. Fluid may then flow in a net upwards direction, optionally through one or more fluid flow channels directing a current flow, to be sterilized by a UV light source before being reintroduced to the surrounding environment. It will be appreciated that various input and output configurations may characterise such a device. For example, an outlet aperture in such a device may be disposed near or in a top surface of the device, and/or may be disposed on one or more sides of the device.

In accordance with various embodiments, a sterilization device may thus comprise an airflow channel in turn comprising a channel wall defining a sterilization airflow volume between an airflow inlet and an airflow outlet. The channel may be any of various configurations, including a linear channel, a serpentine channel, a spiral channel, a combination thereof, or another three-dimensional configuration. For example, air may be taken in through a lower intake aperture from a biofilter purification module disposed below and upstream of the sterilization device within an air purification system. Air may be directed upwards within the sterilization device through a cross-sectionally rounded or rectangular channel, to then be directed outwards and downwards through a fluidically coupled ‘ring’ channel circumscribing the first inlet channel. Regardless of the particular channel configuration, it will be appreciated that the configuration itself may impart or generally define an airflow velocity profile with the channel, a non-limiting example of which is described above with respect to FIG. 6. That is, the geometry and/or configuration of an airflow channel may produce different flow velocities and/or regimes in different regions of the device upon flow of air therethrough. Accordingly, various embodiments relate to the provision of an airflow channel configuration that is designed or selected to provide a designated airflow velocity profile, for instance as a function of a total airflow rate.

Such a device may further comprise a UV light source disposed on an inner surface of one or more of the channel walls so to sterilize at least a portion of the sterilization airflow volume in accordance with a designated spatial irradiance intensity profile. As described above, such sterilization may further be a function of an airflow velocity profile. For example, slower moving air has an increased residence time in a given region of the channel, and is thus irradiated for a longer time. Accordingly, a UV source and/or channel configuration may be provided in consideration of both airflow velocity profiles and a spatial irradiance intensity profile.

For example, a UVC LED may be disposed on the channel wall to emit UVC light in a given volume therearound in an airflow stream at an intensity sufficient to provide sterilization for a given flow rate or residence time. In some such embodiments, the airflow channel may be configured for fluidic communication via the airflow inlet and the airflow outlet with a purification module of the air purification system and the air intake and air output of the air purification system, thus allowing a given volume of air to flow through both a purification module and sterilization module, thereby both sterilizing and purifying previously unsterilized and unpurified air (e.g. ambient air in an office space). It will be appreciated, however, that while some embodiments include an air purification module, that various other embodiments may relate to a standalone sterilization device wherein a UV light source is placed in consideration of irradiance and flow velocity profiles, or similar devices incorporated in or with external airflow systems.

For example, in one embodiment, a UV light source may be disposed on a channel wall in a region corresponding to a higher velocity of an airflow velocity profile, as compared to another region of said sterilization airflow volume. Accordingly, fast moving air, which may not reside in a region for a very long time, may be more heavily irradiated than air at a position further away, which may experience lower irradiation intensity, but for longer. In some embodiments, such considerations may be exploited to increase a uniformity of sterilization of air, and/or the portion of the sterilization airflow volume being irradiated. That is, through placement of the UV light source in consideration of both airflow velocity profiles (e.g. conferred from a channel geometry for a given general airflow rate) and irradiance intensity profiles, sterilization of air across or through a channel region may be improved (e.g. with less air being below a threshold sterilization dosage of UV light, or the like).

In accordance with yet other embodiments, a sterilization device may be used for sterilizing a flow of air in an air purification system comprising an air intake configured to receive unpurified and unsterilized air and an air output configured to allow egress of purified and sterilized air. In some embodiments, the air purification system also comprises an air purification module, an airflow inlet of the sterilization device may be configured to receive air intake from a purification module. That is, the sterilization device may receive from the purification module purified but unsterilized air, to then allow egress of air that is both sterilized and purified. Such a configuration may be beneficial to, for instance, increase a sterilization efficiency of the sterilization device. That is, air that is purified of one or more contaminants may allow for greater sterilization efficiency through more effective propagation of UV light, resulting in a higher level of irradiance, or greater distance from the UV source at which a designated (e.g. sterilizing) irradiance may be achieved. Moreover, such a configuration may reduce maintenance (e.g. cleaning), as purified air may contain less contaminants that may build up on the channel walls upon prolonged exposure.

In some such embodiments, the purification module of such a system may comprise a filter removing a contaminant(s) from ambient air. For example, a filter may remove particles above a certain size. In accordance with another embodiment, a purification module may comprise a biofilter (e.g. a bacterial culture, algae, a plant, or the like), which may preferentially remove certain contaminants (e.g. VOCs). In some such embodiments, the biofilter may comprise a genetically modified organism, such as a genetically modified plant (e.g. one or more ivy and/or tobacco strains) or other organism, such as a genetically modified bacteria or algae, endowed with, for instance, genes to improve its ability to survive or flourish in such an environment, or to improve the plant's ability to remove certain compounds (e.g. VOCs) from air in the surrounding environment.

It will be appreciated that, in accordance with some such embodiments, a sterilization device may further comprise an airflow current generating device, such as a fan or turbine. That is, while some embodiments may rely on airflow currents generated externally (e.g. naturally through convection in an environment, produced by a central air or HVAC unit, or the like), some embodiments provide airflow directly using a corresponding device. It will be appreciated that such a device may be disposed in the sterilization device itself (e.g. at or near an inlet or outlet of the sterilization device), or may be disposed elsewhere in an air purification system to which the sterilization device is fluidically coupled.

As with embodiments described above, some such embodiments of a sterilization device may further comprise a UV-reflective medium disposed on at least a portion of the inner surface of a channel wall. For example, a UV-reflective coating may be applied to the inner surface of the channel wall to minimise risk of UV light escaping the device, even if the base material of the device is not inherently opaque to such wavelengths. This may further increase the efficiency of light propagation and sterilization, as described above. In accordance with other embodiments, a UV-reflective coating may be selectively applied to designated regions of a channel wall to provide and/or govern a designated irradiance intensity profile within a specific region of the device.

It will be appreciated that diverse alternative configurations may similarly be considered within the general scope and nature of the disclosure, and that various aspects herein described, non-limiting examples of which may include the disposition of a light source in a region of fluid flow that is greater in velocity than that of another region of the volume to be sterilized; the use of UVC LEDs; the use of UVC-reflective surfaces; an increased uniformity of an irradiation profile; the shaping of an irradiation intensity profile through the use of beam-shaping elements, such as via the design of a device region(s) comprising a UVC-reflective coating, lenses, mirrors, or other beam shapers; and/or the use of a sterilization device within an air purification system comprising a purification module may be applied to efficiently sterilize a fluid, in accordance with various embodiments.

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Except to the extent necessary or inherent in the processes themselves, no particular order to steps or stages of methods or processes described in this disclosure is intended or implied. In many cases the order of process steps may be varied without changing the purpose, effect, or import of the methods described.

Information as herein shown and described in detail is fully capable of attaining the above-described object of the present disclosure, the presently preferred embodiment of the present disclosure, and is, thus, representative of the subject matter which is broadly contemplated by the present disclosure. The scope of the present disclosure fully encompasses other embodiments which may become apparent to those skilled in the art, and is to be limited, accordingly, by nothing other than the appended claims, wherein any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are hereby expressly incorporated by reference and are intended to be encompassed by the present claims. Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the disclosure.

1. A device for sterilizing a flow of gaseous fluid comprising:
an elongate housing having a quadrilateral perimeter shape with opposing first and second ends, the housing formed from a front panel, a rear panel, a first side panel, a second side panel, a curved bottom panel, and a top panel;
an inner divider coupled to the first and second side panels and extending to near the curved bottom panel for partitioning the interior of the housing into an inlet channel and an outlet channel, wherein the inlet and outlet channels are in fluid communication;
the inner divider having a curved upper end coupled to an interior side of the front panel near the first end above a fluid inlet aperture for guiding fluid into the inlet channel;
at least one fluid outlet aperture located near a downstream region of the outlet channel for allowing fluid to egress from the outlet channel;
at least one UV light source located near a region of higher fluid velocity in the housing as compared to other regions in the housing.

2. The device of claim 1, further comprising at least one fluid current generating device located in the outlet channel near the downstream region of the outlet channel.

3. (canceled)

4. The device of claim 1, wherein the at least one fluid outlet aperture is located in the top panel.

5. (canceled)

6. (canceled)

7. (canceled)

8. (canceled)

9. (canceled)

10. (canceled)

11. (canceled)

12. The device of claim 1, wherein the housing and the inner divider are reflective.

13. (canceled)

14. (canceled)

15. (canceled)

16. The device of claim 1, wherein the inlet and outlet channels have a depth span between the inner divider and the respective front and rear panels and a width span between the first side panel and the second side panel in a ratio of from about 0.1 to about 0.4.

17. (canceled)

18. (canceled)

19. The device of claim 1, further comprising at least one sensor configured to acquire data related to a device parameter, wherein said at least one sensor comprises one or more of a temperature sensor, a humidity sensor, an anemometer, a particular matter sensor, an irradiance sensor, or a volatile organic compound sensor.

20. (canceled)

21. The device of claim 19, further comprising a digital application associated with the device and operable to process said data related to a device parameter acquired by said at least one sensor, wherein said digital application is operable to control one or more of a fluid current generating device or said at least one UV light source to provide an output in response to said acquired data related to a device parameter.

22. (canceled)

23. (canceled)

24. (canceled)

25. (canceled)

26. A device for sterilizing a flow of gaseous fluid comprising:
an elongate housing having one or more sidewalls with opposing first and second ends, a curved bottom panel, and a top panel;
an inner divider coupled to the one or more sidewalls, spanning from one side to the other, and extending to near the curved bottom panel for partitioning the interior of the housing into an inlet channel and an outlet channel, wherein the inlet and outlet channels are in fluid communication;
the inner divider having a curved upper end coupled to an interior side of the one or more sidewalls near the first end above a fluid inlet aperture for guiding fluid into the inlet channel;
at least one fluid outlet aperture located near a downstream region of the outlet channel for allowing fluid to egress from the outlet channel;
at least one UV light source located near a region of higher fluid velocity in the housing as compared to other regions in the housing.

27. The device of claim 26, further comprising at least one fluid current generating device located in the outlet channel near the downstream region of the outlet channel.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. The device of claim 26, wherein the inlet and outlet channels have a depth span between the inner divider and the one or more sidewalls near an apex of the one or more sidewalls and a chord between the edges of the one or more sidewalls defining a width span having a depth span to width span in a ratio of from about 0.1 to about 0.4.

43. (canceled)

44. (canceled)

45. The device of claim 26, further comprising at least one sensor configured to acquire data related to a device parameter, wherein said at least one sensor comprises one or more of a temperature sensor, a humidity sensor, an anemometer, a particular matter sensor, an irradiance sensor, or a volatile organic compound sensor.

46. (canceled)

47. The device of claim 45, further comprising a digital application associated with the device and operable to process said data related to a device parameter acquired by said at least one sensor, wherein said digital application is operable to control one or more of a fluid current generating device or said at least one UV light source to provide an output in response to said acquired data related to a device parameter.

48. (canceled)

49. (canceled)

50. (canceled)

51. (canceled)

52. A sterilization device for sterilizing a flow of air in an air purification system, the sterilization device comprising:
an airflow channel comprising a channel wall defining a sterilization airflow volume between an airflow inlet and an airflow outlet configured to allow respective air intake to and egress from said airflow channel, said airflow channel being configured to impart a designated airflow velocity profile to said sterilization airflow volume; and
a UV light source disposed on an inner surface of said channel wall so to sterilize at least a portion of said sterilization airflow volume in accordance with a designated spatial irradiance intensity profile and said designated airflow velocity profile;
said airflow channel configured for fluidic communication via one or more of said airflow inlet and said airflow outlet with an air intake of the air purification system configured to receive unsterilized air and an air output of the air purification system configured to allow egress of sterilized air.

53. The sterilization device of claim 52, wherein said airflow channel of the sterilization device is further in fluid communication via one or more of said airflow inlet and said airflow outlet with an air purification module of the air purification system.

54. The sterilization device of claim 53, wherein said airflow inlet is configured to receive air intake from said purification module.

55. The sterilization device of claim 53, wherein said purification module comprises a filter.

56. (canceled)

57. The sterilization device of claim 55, wherein said filter comprises a genetically modified organism.

58. (canceled)

59. The sterilization device of claim 52, further comprising a UV-reflective medium disposed on at least a portion of the inner surface of said channel wall.

60. The sterilization device of claim 59, wherein said UV-reflective medium is disposed on the inner surface of said channel wall so to at least partially govern said designated spatial irradiance intensity profile.

61. (canceled)

62. The sterilization device of claim 52, wherein said UV light source is disposed on said channel wall so to provide a uniformity of sterilization of air in said portion of said sterilization airflow volume for said designated spatial irradiance intensity profile and said designated airflow velocity profile.
